(19) United States
(12) Patent Application Publication
(10) Pub. No.: US 2024/0050666 A1
(43) Pub. Date: Feb. 15, 2024

(54) METHODS AND DEVICES FOR MEDICAMENT DELIVERY

(71) Applicant: Palo Alto Research Center Incorporated, Palo Alto, CA (US)

(72) Inventors: Jerome UNIDAD, San Francisco, CA (US); David Mathew JOHNSON, San Francisco, CA (US); Graham ALDINGER, Palo Alto, CA (US); Kevin PINTONG, Palo Alto, CA (US)

(73) Assignee: Palo Alto Research Center Incorporated, Palo Alto, CA (US)

(21) Appl. No.: 17/819,152

(22) Filed: Aug. 11, 2022

(51) Int. Cl. A61M 11/00; A61K 39/12

(52) U.S. Cl. CPC A61M 11/007; A61K 39/12; A61M 2205/8206; A61M 2250/00; A61K 2039/552

(57) ABSTRACT

Mass vaccination of one or more animals may be performed topically using viscosified fluids and a spray delivery device. When applied to one or more animals, the viscosified fluids may adhere a medicament upon a topical surface of the one or more animals. The spray delivery device may comprise at least a spray component, a fluid reservoir component, a plunger, an electronics component, and a battery.

### CROSS-REFERENCE TO RELATED APPLICATIONS

This application is related to U.S. patent application Ser. No. 17/551,289, filed on Dec. 15, 2021. This application is also related to U.S. Pat. No. 11,246,997, patented on Feb. 15, 2022. The disclosures of the above-mentioned application and patent are incorporated herein by reference in their entirety.

### FIELD

The present disclosure generally relates to methods and devices for delivery of medicament to animals.

### STATEMENT REGARDING FEDERALLY SPONSORED RESEARCH OR DEVELOPMENT

Not applicable.

### BACKGROUND

Topical delivery systems for medicaments require a careful selection of both the active component (medicament) and the delivery vehicle in which the active component is dispensed. There is often a correlation between viscosity of the delivery vehicle and 1) the length of time over which the active component remains effective once applied topically, and 2) the ease with which the delivery vehicle may be applied topically via a delivery device. Low-viscosity topical delivery systems typically exhibit lower bioadhesion and may be easily lost from a topical surface. Topical delivery systems having higher viscosity may be held more robustly upon a topical surface, but they may be more difficult to apply due to their higher viscosity, particularly by spraying. Furthermore, some topical delivery systems may contain only a low level of active component, oftentimes due to poor compatibility between the active component and the delivery vehicle.

Uncontrolled disease spread within animal populations may have significant ecological, economic, and public health consequences. Disease spread within and across various animal species may promote evolutionary emergence of new disease strains. The new disease strains may be more transmissible and/or more virulent and pose a significant health hazard when spread from a “reservoir” animal species to humans or another animal species. Such cross-species zoonotic disease spread is believed to be responsible for a number of emerging infectious diseases such as Ebola, Lassa fever, swine flu, and COVID-19. Medicament delivery to animal populations, both in the wild and domestically, may help alleviate these factors and aid in stabilizing an animal population as well. For example, by slowing or stopping disease spread within an animal population, the overall health of the population may improve and increase survival of the population's members. Similarly, immunocontraception vaccination of animal populations may aid in managing overabundant populations of certain wildlife species.

Despite the benefits of administering medicaments to animal populations, there are issues of practicality associated with mass administration of medicaments, especially to animal populations in the wild. To facilitate mass administration of medicaments to an animal population, topical administration is often performed, such as by manual application or through spraying a suitable medicament delivery system. Spray delivery of medicaments may allow multiple animals within an animal population to be treated simultaneously. Once topically applied to an animal's skin, fur, feathers, scales, or the like, the medicament may be directly adsorbed systemically or it may be ingested and subsequently adsorbed as the animals groom themselves or each other. A difficulty with spray delivery approaches is that there are few delivery vehicles that provide a sufficiently low viscosity to promote sprayability, while simultaneously maintaining a high enough viscosity for topical retention (bioadhesion) to occur until the medicament is adsorbed or ingested.

Certain delivery vehicles, such as glycerin jelly, may be too viscous to facilitate effective delivery through spraying. Indeed, even at idealized retention temperatures, the high viscosity of glycerin jelly and similar viscous substances may render spray delivery and retention of medicaments problematic. At the very least, the effective operating temperature range for such delivery vehicles may be rather narrow.

A particular wildlife disease of interest for mass administration of a medicament is white-nose syndrome (WNS), an emergent infectious disease in bat populations that is caused by the fungus Pseudogymnoascus destructans. WNS affects hibernating bats and is considered one of the worst wildlife diseases of modern times, as it has dramatically decreased bat populations. Particularly in the northeastern United States, where cases were first reported, bat populations have been reduced up to 90% in some locations. Moreover, WNS continues to spread across the American continent, with cases now being reported on the West Coast. To date, effective treatments against the fungus that causes WNS have yet to be developed. Moreover, there remain issues with effectively administering vaccines or other medicaments en masse to a bat colony to combat WNS or other infectious diseases.

Given the importance of bats in many ecosystems, the decrease in bat populations is especially concerning. Moreover, the role of bats in spreading emerging zoonotic diseases (e.g., Ebola, Lassa fever, Marburg virus, and coronaviruses, such as COVID-19) in various ecological hot spots is also worrisome from a public health standpoint. Therefore, developing suitable delivery vehicles and techniques to facilitate mass vaccination or treatment of bats against disease may be desirable with respect to both ecological and public health standpoints. While bats are a specific example of a “reservoir” species implicated in zoonotic disease spread, there are other mammalian and non-mammalian species that are also of interest in regard to mass vaccination or treatment to preclude unwanted disease transfer to humans and other animal species.

### SUMMARY

In some aspects, the present disclosure provides a delivery device for medicament delivery within viscosified fluids. The delivery device comprises a spray component, a fluid reservoir component, a plunger, an electronics component, a battery, and optionally a grip and/or a sensor disposed upon a chassis.

In some aspects, the present disclosure provides methods for delivering a medicament to an animal. The methods include spraying a viscosified fluid comprising a medicament onto an animal using a delivery device, the delivery device comprising a spray component, a fluid reservoir component, a plunger, an electronics component, a battery, and optionally a grip and/or a sensor disposed upon a chassis.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 is a diagram of a delivery device for delivery of a viscosified fluid, according to one or more embodiments of the present disclosure.

FIG. 2 is a diagram of a hand-held delivery device for delivery of a viscosified fluid, according to one or more embodiments of the present disclosure.

FIGS. 3-5 are diagrams of inner components of delivery devices for delivery of a viscosified fluid, according to one or more embodiments of the present disclosure.

FIG. 6 is a diagram of multiple delivery devices located atop a cave entrance for delivery of a viscosified fluid, according to one or more embodiments of the present disclosure.

### DETAILED DESCRIPTION

The present disclosure generally relates to methods and devices for delivery of medicament to animals.

As discussed above, there are ongoing challenges associated with mass administration of medicaments to animal populations. Current vaccination of animal populations occurs orally and requires a bait that is attractive to the animals or is otherwise administered via human intervention. Alternatively, topical medicaments may be used, but have various associated issues. Indeed, topical retention of a medicament upon an animal and delivery thereof may be difficult to realize in combination with one another. In the case of mass medicament delivery by spraying, viscous substances that may promote adherence of a medicament to an animal's skin, fur, feathers, scales, or the like may be difficult to spray. However, current spray devices are viscosity-limited and only operate effectively with low-viscosity medicaments, which cannot effectively adhere to an animal's skin, fur, feathers, scales, or the like, which is essential to vaccination treatment and transferability of vaccination fluids between animals (e.g., via grooming or other contact).

The present disclosure describes medicament delivery devices for viscosified fluids that may provide biocompatibility, stability, bioadhesion, and convenient viscosity values for topical delivery to animals. Advantageously, the medicament delivery devices of the present disclosure are able to transmit the viscosified fluids described herein for topical treatment of animals, wherein prior devices are not equipped to successfully spray such viscosified fluids. Moreover, the viscosified fluids of the present disclosure may remain viscous over a range of temperatures commonly encountered in animal habitats (e.g., up to about 40° C.), thus limiting medicament loss as an animal moves between different temperature conditions.

The delivery devices of the present disclosure are designed to “spray” viscosified fluids comprising a medicament onto animals, having a viscosity, for example, of greater than about 1 Pa·s at zero shear. Moreover, the delivery devices may be hand-held or otherwise automated or remotely operated, such that when an animal (e.g., bats) enters or exits their habitat, they can advantageously be sprayed with a medicament fluid without human intervention. As such, the delivery devices of the present disclosure are field-portable and tunable to meet desired spray plume characteristics to allow animal vaccination from a distance, thus requiring no physical touching of the animal.

As indicated herein, the viscosified fluids of the present disclosure are administered to an animal population by spraying. Spraying the medicament, such as a therapeutic agent (drug) or vaccine, may be desirable for mass treatment of an animal population, especially when the animal population is not easily accessible and/or the number of animals is too large to allow individual medicament administration to take place. Animal populations that may be treated with the viscosified fluids disclosed herein are not believed to be particularly limited and may include both domestic animals, such as livestock, and wild animal groups of various types. The wild animals may be mammals, such as bats, prairie dogs, rodents, squirrels, raccoons, cats, dogs, apes, monkeys, and other small or large mammals, birds, reptiles, or the like. Depending on the type of animal, the viscous medicament may stick to the animal's skin, fur, feathers, scales, or the like when sprayed topically using the delivery devices of the present disclosure. The medicament may then be ingested as the animals groom themselves or each other, after which it may impart a therapeutic effect. Alternately, systemic absorption of the medicament through the animal's skin may take place.

All numerical values within the detailed description and the claims herein are modified by “about” or “approximately” with respect to the indicated value, and take into account experimental error and variations that would be expected by a person having ordinary skill in the art. Unless otherwise indicated, ambient temperature (room temperature) is about 25° C.

As used in the present disclosure and claims, the singular article forms “a,” “an,” and “the” include plural forms unless the context clearly dictates otherwise.

The term “and/or” as used in a phrase such as “A and/or B” herein is intended to include “A and B,” “A or B,” “A,” and “B.”

As used herein, the term “shear” refers to stirring or a similar process that induces mechanical agitation to a fluid.

As used herein, the term “aqueous carrier fluid” refers to any fluid containing at least 50 wt. % water.

The delivery devices of the present disclosure are designed to deliver a viscosified fluid comprising a viscosifying construct and a medicament admixed with an aqueous carrier fluid. The viscosifying construct may comprise a polymer associated with a plurality of particles through an ionic interaction, covalent bonding, hydrogen bonding, or any combination thereof. The viscosified fluids may exhibit shear-thinning behavior and may become sprayable once sheared. Shearing may take place before spraying, or the shearing process may take place in concert with the spraying process in some instances.

The spray delivery devices of the present disclosure allow for medicament dose control, plum spray tunability (e.g., spray droplet size, droplet viscosity, splay of spray cone, and the like), and are scalable to a large animal populations. Moreover, they are capable of delivering the viscosified fluids described herein. Differently, oral treatments, needle injection treatments, and traditional topical administration lacking the viscosified fluids of the present disclosure fail to provide one or more of dose control, tunability, and scalability.

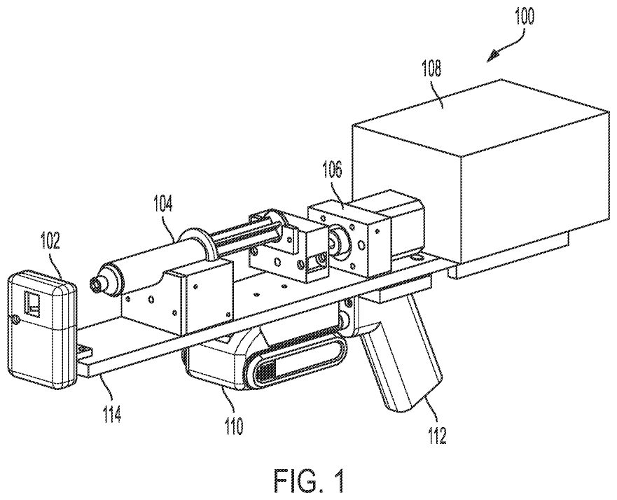

Referring now to FIG. 1, illustrated is a diagram of a suitable delivery device 100 for spraying a viscous medicament onto an animal. As shown, the delivery device 100 comprises a spray component 102 to direct the viscous medicament to a desired target (e.g., animal to be treated), a fluid reservoir component 104 (e.g., syringe) comprising the viscous medicament, and a plunger 106 for ejecting the medicament from the fluid reservoir component 104 and through the spray component 102. The plunger 106 is controlled via an electronics component 108. As shown, the delivery device 100 may further comprise a battery 110 and a grip 112. Each of the spray component 102, the fluid reservoir component 104, the plunger 106, the electronics component 108, the battery 110, and the grip 112 may be disposed upon a chassis 114. Other such elements of the delivery device 100 include screws, bolts, fasteners, pumps, rollers (e.g., one or more), user interface, motors (e.g., pneumatic, air pressure driven, electronic, and the like), and the like, as provided hereinbelow. Moreover, each of the shapes of the various components of delivery device 100 are not particularly limiting and may be altered, without departing from the scope of the present disclosure.

The delivery device 100 may or may not comprise the grip 112, depending upon the particular use of the delivery device. For example, if the delivery device 100 is automated or remotely operated, it needn't include a grip. However, even if automated or remotely operated, the grip 112 may be included to ensure that it can be used manually as well.

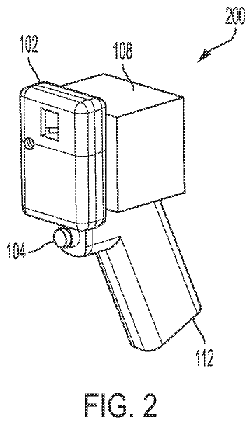

Referring now to FIG. 2, illustrated is a simplified handheld delivery device 200 for spraying a viscous medicament onto an animal, which is similar to delivery device 100 of FIG. 1. Like labeling will be used between FIG. 1 and FIG. 2. As shown, delivery device 200 comprises a spray component 102, electronics component 108, and a grip 112. The viscosified fluid may be comprised within the spray component 102 or the grip 112, for example. For example, a fluid reservoir component 104 may be positioned within an upper portion of the grip 112.

The delivery devices of the present disclosure are capable of spraying viscosified fluids comprising a medicament to an animal, whereas other spraying devices are incapable of delivering such viscous fluids. As such, the bore of the fluid reservoir component 104 through which the viscosified fluid is ejected may have a diameter in the range of about 0.5 millimeters (mm) to about 5 mm, encompassing any value and subset therebetween. The spray component 102 has an opening that is a larger diameter than the particular bore of the fluid reservoir component 104.

As provided above, the delivery devices of the present disclosure may be hand-held or otherwise automated or remotely operated. For example, the delivery devices may be equipped with a sensor that senses the movement of animals into and out of their habitat. As such, the delivery device may be capable of spraying a medicament onto the skin, fur, feathers, scales, or the like, without any human intervention. Thus, other than refilling the medicament, the animal's habitat and living routine is not disturbed. Moreover, the delivery device can be used to confer specific dosages of medicament over an extended animal population, such as due to mating, swarm behavior, or grooming without having to dose each and every animal separately. That is, one or several “index” animals may be treated and thereafter confer protection to other members of the animal population.

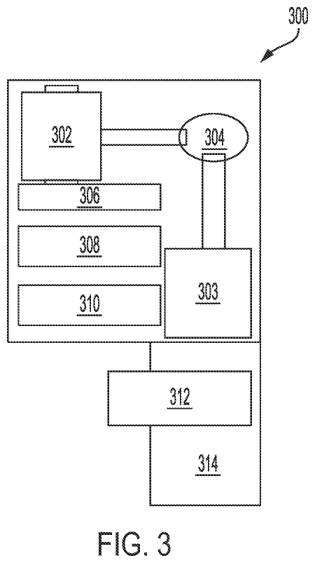

FIGS. 3-5 illustrate various configurations of the delivery devices of FIGS. 1 and 2, including both hand-held and sensor (i.e., automated or remote) driven delivery devices.

Referring first to FIG. 3, illustrated are components of a hand-held delivery device 300 according to one or more aspects of the present disclosure. As shown, the device comprises rollers 302 for delivery of the medicament from a fluid reservoir 303 using a pump 304, a motor 306, a battery 308, control electronics 310, and a user interface 312. The user interface 312 may be a display comprising a touchscreen or otherwise be a simplified manual button, for example. Each of the components of hand-held delivery device 300 are contained within a single body 314.

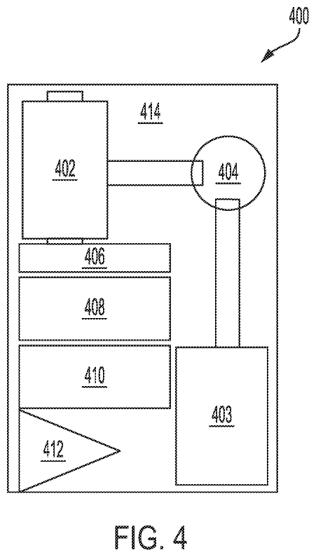

FIG. 4 illustrates components of a sensor-driven delivery device 400, which can be operated upon sensing the movement of a particular animal into and out of its habitat, such as a bat entering or exiting a cave in which it resides. As provided, FIG. 4 comprises the same components of FIG. 3 with the exception of a user interface, which is replaced with a sensor for sensing animal movement. In particular, the delivery device 400 includes rollers 402 for delivery of the medicament from a fluid reservoir 403 using a pump 404, a motor 406, a battery 408, control electronics 410, and a sensor 412. Each of the components shown in FIG. 4 are housed within a single body 414.

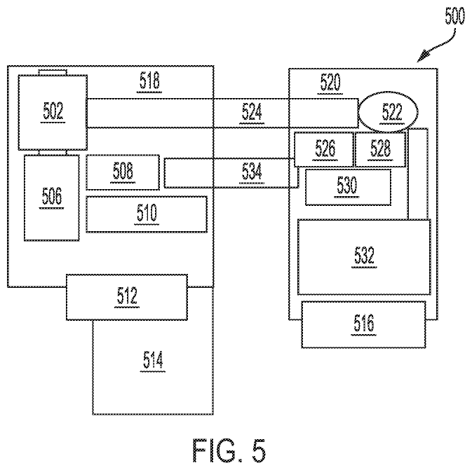

Referring now to FIG. 5, an alternative to a hand-held and/or remotely operated delivery device 500 is provided. In this instance, the delivery device 500 may be positioned upon a positioning location 514, such as a pole or cave entrance or other location in which animals intended to receive the viscosified fluids comprising a medicament traverse. The delivery device 500 may have separate user interfaces (e.g., buttons for operation) 512, 516. The first user interface 512 may be located within body 518 comprising rollers 502, motor 506, battery 508, and control electronics 510. A separate user interface 516 may be located within body 520 that is remotely located from body 518 (e.g., within a backpack, suitcase, or other auxiliary unit), thereby allowing remote operation of the delivery device 500 at a distance from the animals to be treated so as to avoid disturbance of the animals. Moreover, because the body 520 is remotely located, it may comprise a comparatively bigger battery, larger fluid reservoir, and the like.

The body 520 may further comprise a pump 522 that is connected to the rollers 502 via one or more tubes 524. The remote body 520 comprises a battery 526, a motor 528, control electronics 530, and a fluid reservoir 532 connected to the pump 522, tube 524, and spray rollers 502 for delivery of a viscosified fluid comprising a medicament. Each of the electrical components of the bodies 518 and 520 may be connected via electronic wires 534.

The delivery devices of the present disclosure may provide a plume of viscous medicament in an amount of about 0.25 milliliters (mL) to about 1 mL, encompassing any value and subset therebetween, such as about 0.25 mL to about 0.5 mL, or about 0.5 mL to about 1 mL. As such, the plume of sprayed medicament can attach to one or multiple animal's skin, fur, feathers, scales, or the like from a distance. Further, the delivery devices described herein may be equipped with a medicament fluid-filled syringe or other tubular for either single dose or continuous provision of the medicament to animals. In one or more aspects, air or other gas may be used to direct the spray plume and further allow for dose control.

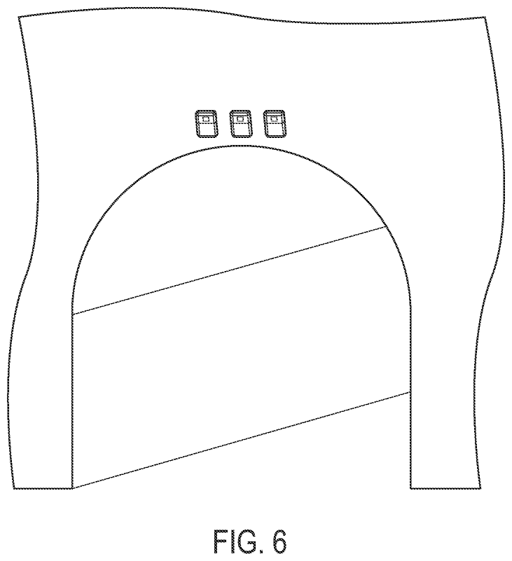

The delivery devices described herein can be used at a distance from an intended animal target and can provide a spray plume to coat at least a portion of the animal. FIG. 6 illustrates an example of multiple (three shown, though more or less may be used) spray components located atop a cave entrance for spraying animals that enter and/or exit the cave.

The splay of the spray plume may have a width in the range of about 5 centimeters (cm) to about 15 cm, encompassing any value and subset therebetween, as determined at the furthest spray location from the spray component.

Suitable medicaments may include any therapeutic agent that may be adsorbed through an animal's skin and/or ingested orally to exert a therapeutic effect. Medicaments may include drugs, such as an anti-inflammatory agent, an antibiotic, an antifungal agent, an antiviral agent, a chemotherapeutic agent, or the like, or a vaccine, for example. The medicament may exert a local or systemic effect when applied topically and subsequently adsorbed or once ingested.

The delivery devices of the present disclosure provide viscosified fluids to small mammals, such as bats, to treat an existing medical condition (disease) may include, but are not limited to, antibiotics, anesthetics, analgesics, antifungals, antihelminthics, and broad spectrum antimicrobials. Representative examples of medicaments among these classes may include, for instance, amoxicillin, amoxicillin-clavulanic acid, enroflaxcin, cephalosporin, clindamycin, doxycycline, isoflurane, tiletamine/zolazepam (TELAZOL), diazepam, ketamine, benzocaine/butamben/tetracaine (CETACAINE), meloxicam, tramadol, buprenorphrine, grifulvin V (micro-sized griseofulvin), chlorhexidine acetate, chlorhexidine gluconate, praziquantel, sulfadimethoxine, fenbendazole, Manuka honey, hypochlorous acid, and the like.

The medicament provided by the delivery devices described herein may be a vaccine in some instances. Unlike other types of medicaments, vaccines may be administered to an animal to prevent a medical condition (disease) from occurring, rather than treating an existing disease. Suitable vaccines may be selected to protect against an inflammatory condition, a bacterial condition, a fungal condition, a viral condition, a cancer, or any combination thereof. For example, the vaccine may be effective to treat white-nose syndrome (WNS) in bats in some instances. In various vaccine applications, the vaccine may induce immunity within a vaccinated animal by presenting key proteins or virions associated with a viral disease. In other instances, vaccines may promote immunity against non-viral diseases (e.g., bacterial infections such as Salmonella or fungal diseases such as WNS). This strategy may be critical for combating WNS in bats, as no antifungal agents have yet been identified that effectively combat the fungus that produces WNS once an infection has occurred.

The viscosified fluid may have a loading of the medicament of about 10 wt. % or less, or about 5 wt. % or less, or about 2 wt. % or less, or about 1 wt. % or less, based on total mass of the viscosified fluid. In the case of a vaccine as a medicament, the viscosified fluid may have a loading of a vaccine ranging from about 10⁴ PFU (plaque forming units) to about 10¹⁰ PFU per 100 μL, of viscosified fluid. In non-limiting embodiments, the medicament may be present at a concentration such that it may be uniformly distributed, either as a dispersion or a solution in an aqueous medium (aqueous carrier fluid) containing the viscosifying construct. A minimum concentration of the medicament in the viscosified fluid may be a therapeutically effective amount sufficient to produce a physiological effect once applied to an animal.

The viscosified fluids may comprise an effective amount of the viscosifying construct to afford a viscosity that is sufficiently low (when sheared or during a post-shearing recovery period) to facilitate administration by spraying and that is sufficiently high (when not sheared or after a post-shearing recovery period) to promote bioadhesion to a topical location upon an animal (e.g., upon the animal's skin, fur, feathers, scales, or the like). In non-limiting embodiments, the viscosifying construct may be present in an aqueous carrier fluid at a concentration such that the plurality of particles is present below a percolation threshold of the particles alone. For both particles and polymer separately, the percolation threshold is defined by a critical concentration (c*), also known in the art as the overlap concentration, wherein adjacent particles contact each other. The critical concentration (c*) may depend upon the dimensions of the particle or the length of polymer chains. In the case of cellulose particles having a largest dimension of about 1 micron, the critical concentration may occur at a concentration of about 5 wt. % or less. By having the particles in the viscosifying construct present below the percolation threshold, potential toxic effects associated with higher particle concentrations may be lessened.

In non-limiting examples, the loading of polymer in the viscosified fluids may range from about 0.001 wt. % to about 30 wt. %, encompassing any value and subset therebetween, such as about 0.001 wt. % to about 20 wt. %, or about 0.01 wt. % to about 20 wt. %, or about 0.1 wt. % to about 20 wt. %, or about 1 wt. % to about 20 wt. %, or about 1 wt. % to about 15 wt. %, or about 1.5 wt. % to about 10 wt. %, or about 2 wt. % to about 5 wt. %, based on the total mass of the viscosified fluids.

In non-limiting examples, the loading of particles in the viscosified fluids may range from about 0.005 wt. % to about 80 wt. %, encompassing any value and subset therebetween, such as about 0.005 wt. % to about 75 wt. %, or about 0.005 wt. % to about 70 wt. %, or about 0.005 wt. % to about 65 wt. %, or about 0.005 wt. % to about 60 wt. %, or about 0.05 wt. % to about 60 wt. %, or about 0.5 wt. % to about 60 wt. %, or about 1 wt. % to about 60 wt. %, or about 2 wt. % to about 55 wt. %, or about 5 wt. % to about 50 wt. %, or about 10 wt. % to about 60 wt. %, or about 15 wt. % to about 55 wt. %, or about 20 wt. % to about 50 wt. %, or about 25 wt. % to about 45 wt. %, or about 30 wt. % to about 35 wt. %, based on the total mass of the viscosified fluids. Some of these particle loadings may be below the particle percolation threshold, as referenced above.

The viscosifying constructs of the present disclosure may have a mass ratio of particles to polymer ranging from about 1:10 to about 10:1, encompassing any value and subset therebetween, such as about 1:10 to about 1:1, or about 1:5 to about 5:1, or about 1:1 to about 10:1.

The viscosifying constructs may be present in the viscosified fluids in an amount sufficient to produce a viscosity of at least about 1 Pa·s, such as from about 1 Pa·s to about 100 Pa·s, encompassing any value and subset therebetween, such as about 10 Pa·s to about 90 Pa·s, or about 20 Pa·s to about 80 Pa·s, or about 25 Pa·s to about 70 Pa·s, or about 30 Pa·s to about 60 Pa·s at zero shear and at a temperature up to about 40° C. Higher viscosity values may be measured at lower temperatures. As used herein, viscosity of the viscosified fluids are measured at either 20° C. or 40° C. using strain-controlled shear rheometry, in which the rheometer is equipped with parallel plates.

In some embodiments, the polymer and the particles may comprise nucleophilic or electrophilic functional groups, wherein a first functional group upon the polymer is nucleophilic or electrophilic and a second functional group upon the particles is the other of nucleophilic or electrophilic. For example, if the polymer has nucleophilic functional groups such as amines, the particles may comprise electrophilic functional groups, such as N-hydroxysuccinimides, to promote covalent bond formation. Likewise, if the polymer has electrophilic functional groups such as sulfosuccinimides, the particles may have nucleophilic functional groups such as amines, thiols, or carboxylic acids.

In some embodiments, the polymer and the particles may comprise hydrogen bond donors or hydrogen bond acceptors. For example, a first functional group upon the polymer may be a hydrogen bond donor, and a second functional group upon the particles may be a hydrogen bond acceptor.

The polymer and the particles may comprise functional groups with opposite charges, which may promote salt bridge formation or a similar type of ionic interaction. More specifically, the polymer and the particles may be associated with each other through an ionic interaction in which the first functional group and the second functional group bear opposite charges. In more particular examples, the polymer may be positively charged and the particles may be negatively charged. For example, the polymer may comprise positively charged amine groups, and the particles may comprise negatively charged sulfate groups, sulfonate groups, and/or carboxylate groups. Thus, functional polymers such as proteins, poly(allyl amine), or amine-terminated di- or multifunctional poly(ethylene glycol) (PEG) may be used as the polymer in the disclosure herein in combination with particles bearing sulfate, sulfonate, carboxylate, or phosphate groups. Other suitable examples of polymers and particles are described further below.

Particles suitable for incorporation in the viscosifying constructs are not believed to be particularly limited, provided that they are biocompatible and contain suitable functional groups for pairing with those upon a polymer or an intermediate species. Suitable particles may include, for example, minerals, silicates, synthetic clays, biopolymers, and any combination thereof. The particles may be native or have undergone chemical modification (e.g., thermal reduction, hydrolysis, sulfonation, or the like). The particles may be isotropic (e.g., true particles, spheres, or precipitates) or anisotropic (e.g., platelets, rods, fibrils, or the like). The particles may be of any size range and may comprise nanoparticles in particular embodiments. Suitable sizes may range from about 1 nm up to about 1 micron in a largest dimension of the particles.

In particular embodiments, the particles may comprise cellulose nanoparticles (CNP) that are functionalized with at least one acid group. CNP (e.g., cellulose nanocrystals) are a class of bio-based nanoscale materials, which are of interest due to their biocompatibility, biodegradability, and renewability. CNP may be categorized into two major classes, (a) nanostructured materials (including cellulose microcrystals and cellulose microfibrils) and (b) nanofibers (including cellulose nanofibrils, cellulose nanorods, cellulose nanocrystals, and bacterial cellulose).

Cellulose nanoparticles, such as cellulose nanocrystals, suitable for use in the disclosure herein may be prepared by any suitable technique known to persons having ordinary skill in the art. Cellulose nanoparticles bearing at least one acid group may be prepared by acid hydrolysis of cellulose, followed by freeze-drying. The cellulose may be obtained from any suitable source, such as cotton or biomass. Hydrolysis using sulfuric acid may introduce sulfate groups to the surface of the cellulose nanoparticles, thereby facilitating their use in the disclosure herein, as well as permitting their dispersion in both aqueous and organic fluids, including water, ethanol and N,N-dimethylformamide. Carboxylate groups may be introduced through oxidation to the surface of the cellulose nanoparticles in some instances.

Alternately, the surface of cellulose nanoparticles may be modified with various electrophiles to promote covalent bond formation with a nucleophilic functional group upon the polymer. For example, the cellulose nanoparticles may be modified to introduce electrophilic a-bromoesters to the particle surface, which may then undergo a reaction with a nucleophile to promote direct covalent bond formation with a complementary functional group upon the polymer (e.g., a thiol, an amine, a carboxylate, a phosphine, or the like) that may vary in hydrophobicity, functionality, and complexity.

Suitable particles for use in the disclosure herein may have a maximum size of about 1 micron in a largest dimension thereof. For example, fibrils may be about 1 micron in length but have other dimensions that are much smaller, such as about 100 nm or less in other dimensions. Particle size can be determined by scanning electron microscopy or dynamic light scattering techniques.

In some instances, the polymer may comprise an amine-containing carbohydrate, such as chitosan. Chitosan has a carbohydrate backbone structure similar to cellulose, which includes two types of repeating units, N-acetyl-D-glucosamine and D-glucosamine, linked by (1-4)-β-glycosidic linkages. Chitosan has been widely used in the pharmaceutical industry due to its biodegradability and biocompatibility properties. Chitosan is a cationic natural polysaccharide exhibiting antimicrobial and antifungal activities, which makes it a favorable excipient option for biomedical applications. Viscosified fluids containing a viscosifying construct formed from chitosan may be especially advantageous for treating white-nose syndrome due to the fungal nature of this condition. In addition, the amine groups of chitosan provide complementary functionality for association via an ionic interaction with negatively charged functional groups upon particles, such as cellulose nanoparticles functionalized with an acidic group, when forming the viscosifying constructs disclosed herein.

Chitosan may be obtained through deacetylation of chitin, often obtained from the shells of shrimp and other sea crustaceans, using excess aqueous sodium hydroxide. Chitosan is soluble in dilute acidic solutions of acetic, citric, and tartaric acids at a pH less than about 6.5, with solubility at about 1-3 wt. % being realized through protonation of the amine group. Suitable chitosans for forming a viscosifying construct may have molecular weight values (Mw) ranging from about 3,800 to about 190,000 Da, encompassing any value and subset therebetween, and with different degrees of deacetylation.

Depending on the functional groups present on the particles, chitosan derivatives may also be suitable for use in the disclosure herein. Suitable chitosan derivatives may include N-alkyl chitosans, thiolated chitosans, carboxylated chitosans, chitosan succinate, chitosan phthalate, mono-N-carboxymethyl chitosan (MCC), or any combination thereof. Other carbohydrate derivatives bearing suitable functionality for interacting with a given functional group upon the particles may also be suitably used in the disclosure herein.

Other types of polymers may also be used in the disclosure herein, provided that they contain or are derivatized to contain one or more functional groups that are complementary with one or more functional groups upon the particles. Suitable alternative polymers may include medical grade synthetic polymers such as, but not limited to, amino-acid-based polymers, polyamides, polyimides, polyurethanes, cellulose-based polymers, starch-based polymers, water-insoluble biodegradable polymers, alginates and other hydrocolloid-based polymers, and any combination thereof. Biocompatible polyamides and polyurethanes may be polyamides and polyurethanes containing a phospholipid moiety.

Other examples of suitable polymers that may be used in the disclosure herein include, for example, poly(ethylene glycol) (PEG), poly(propylene glycol) (PEG), polylactic acid (PLA), polyglycolic acid (PGA), poly (DL-lactide-co-glycolide) (PLG), polyanhydrides, polyacrylic acid, polymethyl acrylates, poly(c-caprolactone) (PCL), poly(3-hydroxybutyrate-co-3-hydroxyvalerate) (PHBV), any copolymer thereof, or any combination thereof.

A first functional group upon the polymer and a second functional group upon the particles may be associated with each other through an intermediate species that is a crosslinking agent. Polymers may be crosslinked with a plurality of particles via an ionic interaction, a covalent bond, a hydrogen bond, or any combination thereof. A crosslinking agent may be employed when a first functional group upon the polymer is not directly complementary with a second functional group upon the particles. The crosslinking agent may be bifunctional and bear at least two types of functional groups that are complementary with the first functional group upon the polymer and the second functional group upon the particles, thereby allowing a network structure to form effectively in the presence of the crosslinking agent. Nonlimiting examples of suitable crosslinking agents may include transition metal salts, borates, boronic acids, bifunctional crosslinkers, and any combination thereof, provided that the crosslinking agent is sufficiently biocompatible once incorporated in a viscosifying construct. A crosslinking agent may facilitate association between a polymer and particles that have the same charge or a different charge, or between a charged polymer and uncharged particles or vice versa.

When used, crosslinking agents may be present in the viscosified fluids disclosed herein in an amount ranging from about 5 wt. % to about 70 wt. %, encompassing any value and subset therebetween, such as about 10 wt. % to 60 wt. %, or about 15 wt. % to 50 wt. %, or about 20 wt. % to 45 wt. %, based on total mass of the viscosified fluid.

Borate crosslinking agents may facilitate crosslinking between alcohols and amines. More particularly, suitable borate crosslinking agents may form borate ester bonds with alcohol functional groups, and the amine group may form a B—N bond through donation of a lone pair of electrons to the unoccupied p-orbital upon the boron atom. Examples of borate crosslinking agents may include, but are not limited to, boric acid, alkali or alkaline earth metal borates, such as disodium octaborate tetrahydrate or sodium diborate, and organic borates such as boronic acids and esters.

Suitable bifunctional crosslinking agents may contain the same functional group (if the first and second functional groups upon the polymer and the particles are the same) or different functional groups (if the first and second functional groups upon the polymer and particles are different). Suitable bifunctional crosslinking agents that may enable linkage via a thioether bond include N-succinimidyl-4-(N-maleimidomethyl)-cyclohexane-1-carboxylate (SMCC) to introduce maleimido groups, or with N-succinimidyl-4-(iodoacetyl)-aminobenzoate (SIAB) to introduce iodoacetyl groups. Other bifunctional crosslinking agents that may introduce maleimido groups or haloacetyl groups include, but are not limited to, bis-maleimidopolyethyleneglycol (BMPEO), BM(PEO)₁, BM(PEO)₃, N-(3-maleimidopropyloxy)succinimide ester (BMPS), γ-maleimidobutyric acid N-succinimidyl ester (GMBS), ε-maleimidocaproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproate), which is a “long chain” analog of SMCC (LC-SMCC), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido)propionate (SBAP), N-succinimidyl iodoacetate (SIA), κ-maleimidoundecanoic acid N-succinimidyl ester (KMUA), N-succinimidyl 4-(p-maleimidophenyl)-butyrate (SMPB), succinimidyl-6-(3-maleimidopropionamido)hexanoate (SMPH), succinimidyl-(4-vinylsulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfosuccinimide ester (sulfo -MBS), N-(γ-maleimidobutryloxy) sulfo suc cinimde ester (sulfo-GMBS), N-(ε-maleimidocaproyloxy)sulfosuccimido ester (sulfo-EMCS), N-(κ-maleimidoundecanoyloxy)sulfosuccinimide ester (sulfo-KMUS), and sulfosuccinimidyl 4-(p-maleimidophenyl)butyrate (sulfo-SMPB).

Heterobifunctional crosslinking agents are bifunctional crosslinking agents having two different reactive groups. Heterobifunctional crosslinking agents may contain both an amine-reactive N-hydroxysuccinimide group (NHS group) and a carbonyl-reactive hydrazine group. Examples of commercially available heterobifunctional crosslinking agents may include, but are not limited to, succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Examples of bifunctional crosslinking agents that can be used include succinimidyl-p-formyl benzoate (SFB) and succinimidyl-p-formylphenoxy acetate (SFPA).

Bifunctional crosslinking agents that enable a linkage via disulfide bonds may include N-succinimidyl-3 -(2 -pyridyldithio)propionate (SPDP), N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to introduce dithiopyridyl groups. Alternatively, crosslinking agents such as 2-iminothiolane, homocysteine thiolactone or S-acetylsuccinic anhydride that introduce thiol groups can also be used.

Suitable crosslinking agents may further include diallyl fumarate, diallyl diglycol carbonate, allyl methacrylate, diallyl phthalate, diallyl suverate, diallyl tetrabromophthalate, diethylene glycol diacrylate, diethylene glycol dimethacrylate, diethylene glycol divinyl ether, N,N′-dimethacryloylpiperazine, 2,2-dimethylpropanediol dimethacrylate, dipentaerythritol pentaacrylate, dipropylene glycol dimethacrylate, ditrimethylolpropane tetraacrylate, divinyl glycol, divinyl sebacate, glycerol trimethacrylate, 1,5-hexadiene, 1,6-hexanediol diacrylate, 1,6-hexane Diol diacrylate, 1,6-hexanediol dimethacrylate, N,N′-methylenebismethacrylamide, 1,9-nonanediol dimethacrylate, pentaerythritol tetraarc Relate, pentaerythritol triacrylate, pentaerythritol triallyl ether, 1,5-pentanediol dimethacrylate, poly (propylene glycol) dimethacrylate, tetraethylene glycol dimethacrylate, triethylene glycol di Acrylate, triethylene glycol dimethacrylate, triethylene glycol divinyl ether, 1,1,1-trimethylolethane trimethacrylate, 1,1,1-trimethylolpropane diallyl ether, 1,1,1-Trimethylolpropane triacrylate, 1,1,1-trimethylolpropane trimethacrylate, tripropylene glycol diacrylate, 1,2,4-trivinylcyclohexane, divinyl benzene, bis (2-methacryl oxyethyl) phosphate, 2,2-bis (4-methacryloxyphenyl) propane, 1,3-butanediol diacrylate, 1,4-butanediol diacrylate, 1,3-butanediol dimethacrylate, 1,4-butanediol dimethacryl Rate, 1,4-butanediol divinyl ether, 1,4-cyclohexanediol dimethacrylate, bis [4-(vinyloxy) butyl] isophthalate, bis [4-(vinyloxymethyl) cyclohexylmethyl] glycol Rutarate, bis [-(vinyloxy) butyl] succinate, bis ((4-((vinyloxy) methyl) cyclohexyl) methyl) isophthalate, bis (4-(vinyloxy) butyl) terephthalate, bis [[(4-(vinyloxy) methyl) cyclohexyl] methyl] terephthalate, bis [4-(vinyloxy) butyl] adipate, bis [4-(vinyloxy) butyl] (methylenedi-1,4-phenylene) biscarbamate, bis [4-(vinyloxy) butyl] (4-methyl-1,3-phenylene) biscarbamate, bis [4-(vinyloxy) butyl] 1,6-hexanediylbis Carbamate or tris [4-(vinyloxy) butyl] trimelitate.

As discussed above, a first functional group upon the polymer may be directly associated with a second functional group upon the particles. In this case, the polymer or particles act as a crosslinker. Hence, when a crosslinking agent or other intermediate species is absent, the functional groups are oppositely charged or otherwise complementary to form a covalent bond or hydrogen bonding interaction.

The viscosified fluids of the present disclosure may further include stabilizing agents, surfactants and/or adjuvants. Examples of surfactants may be any non-toxic, biocompatible and biodegradable surfactants. Examples of suitable surfactants may include, but are not limited to, zwitterionic, neutral, cationic, or anionic surfactants. Common hydrophilic head groups of ionic or zwitterionic surfactants are carboxylate, sulfate, sulfonate, carboxybetaine, sulfobetaine, and quaternary ammonium. Suitable neutral surfactants may include, but are not limited to, lipid-based surfactants, polyethylene glycol (PEG) block-copolymer surfactants, polysorbates, such as TWEEN 20 and TWEEN 80, other sorbitan derivatives, such as sorbitan laurate, and alkoxylated alcohols, such as laureth-4.

Alternately, the viscosified fluids of the present disclosure may be surfactant-free. A surfactant-free viscosified fluid may be desirable when the surfactant interacts in a medicament in an undesired way. For example, surfactants may change the conformation of protein-based medicaments or interfere with a lipid- or micelle-encapsulated medicament.

Methods of the present disclosure may comprise: topically spraying a medicament within a viscosified fluid onto an animal using the delivery devices of the present disclosure. The viscosified fluid is applied topically upon at least one animal. Once sprayed topically, the viscosified fluid is able to stick to the skin, fur, feathers, or scales of the animal, depending on the type of animal being treated, thereby facilitating medicament delivery and retention. The medicament may be absorbed through the skin of the animal, or preferably ingested by the animal as the animal grooms itself or a group of animals grooms each other.

In some embodiments, the animal may be at least one mammal. Mammals that may be treated with the viscosified fluids described herein are not believed to be particularly limited and may include, for example, bats, prairie dogs, rodents, raccoons, livestock animals (e.g., cows, horses, goats, sheep, and the like) cats, dogs, apes, and other small or large mammals, wild or domesticated. The viscosified fluid may be applied to individual animals or to multiple animals simultaneously.

Embodiments disclosed herein include:
A. A delivery device for spraying a viscosified fluid comprising a medicament, the delivery device comprising: a spray component; a fluid reservoir component; a plunger; an electronics component; and a battery.
B. A method comprising: topically spraying a viscosified fluid comprising a medicament onto at least one animal using a delivery device, the delivery device comprising: a spray component; a fluid reservoir component; a plunger; an electronics component; and a battery.

Embodiments A and B may have one or more of the following additional elements in any combination, without limitation:

Element 1: Wherein the delivery device further comprises a sensor.

Element 2: Wherein the delivery device further comprises a grip.

Element 3: Wherein the delivery device further comprises a user interface.

Element 4: Wherein the fluid reservoir component has a bore, the bore having a diameter in the range of about 0.5 millimeters to about 5 millimeters.

Element 5: Wherein the fluid reservoir component has a bore, the bore having a diameter in the range of about 0.5 millimeters to about 5 millimeters, and wherein the spray component has an opening that has a diameter in the range of about 5% to about 10% larger in diameter than the bore.

Element 6: Wherein the medicament comprises a vaccine.

Element 7: Wherein the medicament comprises a vaccine.

Element 8: Wherein the medicament comprises a vaccine and the vaccine is selected from the group consisting of a virus-vectored vaccine, a virus-like particle, a free antigen, a sub-unit antigen, a liposome-formulated nucleic acid vaccine, and any combination thereof.

Element 9: Wherein the viscosified fluid comprises a viscosifying construct and the medicament admixed with an aqueous carrier fluid, the viscosifying construct comprising a polymer associated with a plurality of particles through an ionic interaction, covalent bonding, hydrogen bonding, or any combination thereof, and wherein the viscosified fluid exhibits shear-thinning behavior.

Element 10: Wherein the viscosified fluid comprises a viscosifying construct and the medicament admixed with an aqueous carrier fluid, the viscosifying construct comprising a polymer associated with a plurality of particles through an ionic interaction, covalent bonding, hydrogen bonding, or any combination thereof, and wherein the viscosified fluid exhibits shear-thinning behavior, and wherein the polymer and the plurality of particles comprising the viscosifying construct are present in the aqueous carrier fluid at a concentration of about 5 vol. % or below.

Element 11: Wherein the viscosified fluid comprises a viscosifying construct and the medicament admixed with an aqueous carrier fluid, the viscosifying construct comprising a polymer associated with a plurality of particles through an ionic interaction, covalent bonding, hydrogen bonding, or any combination thereof, and wherein the viscosified fluid exhibits shear-thinning behavior, and wherein the viscosifying construct is present in the aqueous carrier fluid in an amount sufficient to produce a viscosity of at least about 1 Pa·s at zero shear and at a temperature up to about 40° C., as determined by steady shear rheometry.

Element 12: Wherein the viscosified fluid is sprayed upon multiple animals simultaneously.

Element 13: Wherein the at least one animal comprises at least one mammal.

Element 14: Wherein the at least one animal comprises at least one mammal selected from the group consisting of a bat, a prairie dog, a raccoon, a livestock animal, a cat, and a dog.

To facilitate a better understanding of the present disclosure, the following examples of preferred or representative embodiments are given. In no way should the following examples be read to limit, or to define, the scope of the invention.

### EXAMPLES

Viscosified fluids were prepared as specified in Table 1, and viscosity values were determined at 20° C. and 40° C. using shear rheometry.

TABLE 1

| | Viscosity | Viscosity |
|---|---|---|
| Formulation | (20° C.) | (40° C.) |
| 2 wt. % cellulose nanocrystals in water | 0.36 | 0.24 |
| 2 wt. % chitosan in water | 1.24 | 0.54 |
| 1 wt. % cellulose nanocrystals/1 wt. % | 2.49 | 1.56 |
| chitosan in water | | |

As shown, the viscosity values were higher when particles and polymer were combined together in a viscosifying construct, in comparison to a higher concentration of particles or polymer alone. The viscosification effect was more than additive at equivalent total concentrations of polymer and particles.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

What is claimed is the following:

1. A delivery device for spraying a viscosified fluid comprising a medicament, the delivery device comprising:
a spray component;
a fluid reservoir component;
a plunger;
an electronics component; and
a battery.

2. The delivery device of claim 1, further comprising a sensor.

3. The delivery device of claim 1, further comprising a grip.

4. The delivery device of claim 1, further comprising a user interface.

5. The delivery device of claim 1, wherein the fluid reservoir component has a bore, the bore having a diameter in the range of about 0.5 millimeters to about 5 millimeters.

6. The delivery device of claim 5, wherein the spray component has an opening that has a diameter in the range of about 5% to about 10% larger in diameter than the bore.

7. A method comprising:
topically spraying a viscosified fluid comprising a medicament onto at least one animal using a delivery device, the delivery device comprising:
a spray component;
a fluid reservoir component;
a plunger;
an electronics component; and
a battery.

8. The method of claim 7, wherein the delivery device further comprises a sensor.

9. The method of claim 7, wherein the delivery device further comprises a grip.

10. The method of claim 7, wherein the delivery device further comprises a user interface.

11. The method of claim 7, wherein the fluid reservoir component has a bore, the bore having a diameter in the range of about 0.5 millimeters to about 5 millimeters.

12. The method of claim 11, wherein the spray component has an opening that has a diameter in the range of about 5% to about 10% larger in diameter than the bore.

13. The method of claim 7, wherein the viscosified fluid comprises a viscosifying construct and the medicament admixed with an aqueous carrier fluid, the viscosifying construct comprising a polymer associated with a plurality of particles through an ionic interaction, covalent bonding, hydrogen bonding, or any combination thereof, and wherein the viscosified fluid exhibits shear-thinning behavior.

14. The method of claim 13, wherein the polymer and the plurality of particles comprising the viscosifying construct are present in the aqueous carrier fluid at a concentration of about 5 vol. % or below.

15. The method of claim 13, wherein the viscosifying construct is present in the aqueous carrier fluid in an amount sufficient to produce a viscosity of at least about 1 Pa·s at zero shear and at a temperature up to about 40° C., as determined by steady shear rheometry.

16. The method of claim 7, wherein the medicament comprises a vaccine.

17. The method of claim 16, wherein the vaccine is selected from the group consisting of a virus-vectored vaccine, a virus-like particle, a free antigen, a sub-unit antigen, a liposome-formulated nucleic acid vaccine, and any combination thereof.

18. The method of claim 7, wherein the viscosified fluid is sprayed upon multiple animals simultaneously.

19. The method of claim 7, wherein the at least one animal comprises at least one mammal.

20. The method of claim 19, wherein the at least one mammal comprises a mammal selected from the group consisting of a bat, a prairie dog, a raccoon, a livestock animal, a cat, and a dog.
